(19) United States
(12) Patent Application Publication
(10) Pub. No.: US 2014/0303537 A1
(43) Pub. Date: Oct. 9, 2014

(54) KIT FOR CREATING A LUMBAR SUPPORT BELT

(71) Applicant: GIBAUD, Saint Etienne (FR)

(72) Inventors: Guillaume Duport, Saint Etienne (FR); Frederic Girard, Paris (FR)

(21) Appl. No.: 14/356,738

(22) PCT Filed: Oct. 29, 2012
(86) PCT No.: PCT/FR2012/052491

§ 371 Date: May 7, 2014

(30) Foreign Application Priority Data
Nov. 7, 2011 (FR) 11/60129

(51) Int. Cl. A61F 5/02

(52) U.S. Cl. CPC A61F 5/028
USPC 602/19

(57) ABSTRACT

The kit for creating a lumbar support belt includes a dorsal element intended to be placed against the lumbar region of an individual, and comprising reinforcing members; a set of strips of elastic textile that can be positioned around the lower part of the trunk of the individual and comprises assembly means able to collaborate removably with complementary assembly means formed on the dorsal element. Each of the strips has a certain number of structural characteristics, with at least one structural characteristic differing from one strip to another within the set of strips. The kit makes it possible to create a lumbar support girdle comprising the dorsal element and a single strip chosen from the set of strips according to the structural characteristic desired.

The present invention relates to a kit for creating a lumbar support belt.

Such a belt, intended for the support of the lumbar region, is placed around the waist of a user and exerts an appropriate compression allowing to relieve certain aches or prevent their appearance. This belt is usually composed of an elastic textile strip which, to ensure a good hold, is widened in the back region of the patient and is provided with stays.

This strip requires important thus expensive manufacturing operations. In fact, its creation implies in particular steps of cutting, binding and forming gussets wherein the stays will be engaged. Some belts are created upon request for a given patient.

Furthermore, for a perfect adaptation to his/her needs, the wearer should ideally have different belts. In fact, the restraint and/or the geometry of the belt should be adapted according to the development of the pathology and/or according to the activity performed by the wearer.

It follows that known belts do not entirely fulfill the needs of users, unless the latter allocate a significant budget for them.

The purpose of the present invention is to remedy to the aforementioned drawbacks.

To this end, the invention relates to a kit for creating a lumbar support belt, this kit comprising:
on the one hand, a dorsal element intended to be placed against the lumbar region of an individual, said dorsal element comprising at least two reinforcing members; and on the other hand, a set of N strips of elastic textile distinct from the dorsal element, where N≧2, each of the strips being able to be positioned around the lower portion of the trunk of the individual and comprising:
a central area provided with assembly means able to removably cooperate with complementary assembly means formed on the dorsal element;
first and second extreme portions provided with complementary fastening means.

Moreover, according to the invention, each of the N strips has a certain number of structural characteristics, at least one structural characteristic differing from one strip to another within the set of N strips, the kit making it possible to create a lumbar support belt comprising said dorsal element and a single strip chosen from the set of N strips according to said structural characteristic desired.

The invention hence proposes to provide a belt in two portions, wherein the elements making the manufacturing complex being transferred onto the dorsal element. Thus, this dorsal element bears the reinforcing members, that is to say, usually the stays, and has a height sufficient to provide a good support in the lumbar region. Consequently, the strip may be very simplified, the functions thereof being substantially to ensure restraint and provide the fastening means for placing the belt around the patient. Thus, it is provided a strip of extremely simple structure, for example having a constant width and without reinforcing members.

More particularly, by providing a kit comprising a single dorsal element and N strips, the manufacturing costs are considerably reduced with respect to N belts of the prior art, since the most expensive operations are only made on one single piece, namely the dorsal element, and not on each of the strips. Of course, the invention is even more advantageous if the method for manufacturing the dorsal element is simple and not costly.

It is worth noting that, according to the invention, the main purpose of the dorsal element is not restraint as would be the case of a corset, and that the strip cannot be used alone, without the dorsal element.

In practice, the strip is assembled on the external side of the dorsal element and tightens this dorsal element against the body of the wearer. The first and second extreme portions of the strip are placed on top of each other while facing the abdominal region of the wearer and are assembled by the complementary fastening means.

The dorsal element is preferably designed to be able to be used in all situations and to be suitable for all morphologies, thus making it possible to have a single element, or even a single size. As for the strip, it is chosen from the set of N strips according to the needs of the wearer, according to the most appropriate structural characteristic.

A structural characteristic different from one strip to another within the set of N strips may comprise:
the average nervousness of the strip;
the variation of the nervousness in the strip
and/or the geometry of the strip.

Furthermore, at least one of the strips from the set of N strips may comprise an additional tightening device.

According to an advantageous embodiment, the dorsal element may comprise a body obtained by cutting then thermoforming a panel of material, for example a panel of foam. The dorsal element may thus be obtained in a very simple and cheap manner, and thus even if it includes a certain number of functions which, in the prior art, were present on the strip.

In order to improve the breathability of the belt, the dorsal element may comprise a body made from a breathable material and/or provided with orifices.

The dorsal element may comprise at least one cavity for receiving a reinforcing member, said cavity opening onto the external side of the dorsal element and being blocked by a layer secured to the dorsal element. In the case where this cavity is achieved by thermoforming the body of the dorsal element, the creation of the belt is considerably simplified with respect to the prior art. The reinforcing members are thus integrated in the dorsal element and not in the strip of the belt anymore.

According to a possible embodiment, the dorsal element comprises on its internal side at least one relief intended to limit the sliding of said dorsal element with respect to the body of the wearer in at least one direction, preferably the vertical direction in use position. It may also be provided at least one relief intended to limit the sliding of said dorsal element with respect to the body of the wearer in two orthogonal directions, usually vertically and horizontally.

Furthermore, the dorsal element may exhibit a substantially vertical groove, in use position, intended to be placed facing the vertebral column of the wearer. This groove allows ensuring a placing of the dorsal element against the back of the wearer despite the protruding backbone.

It may be provided that the removable assembly means be arranged in order to allow adjusting the position of the strip in height with respect to the dorsal element. The removable assembly means may further comprise visual references able to indicate at least a relative positioning in height of the strip with respect to the dorsal element.

It is now described, by way of non limiting example, several possible embodiments of the invention, with reference to the accompanying figures:

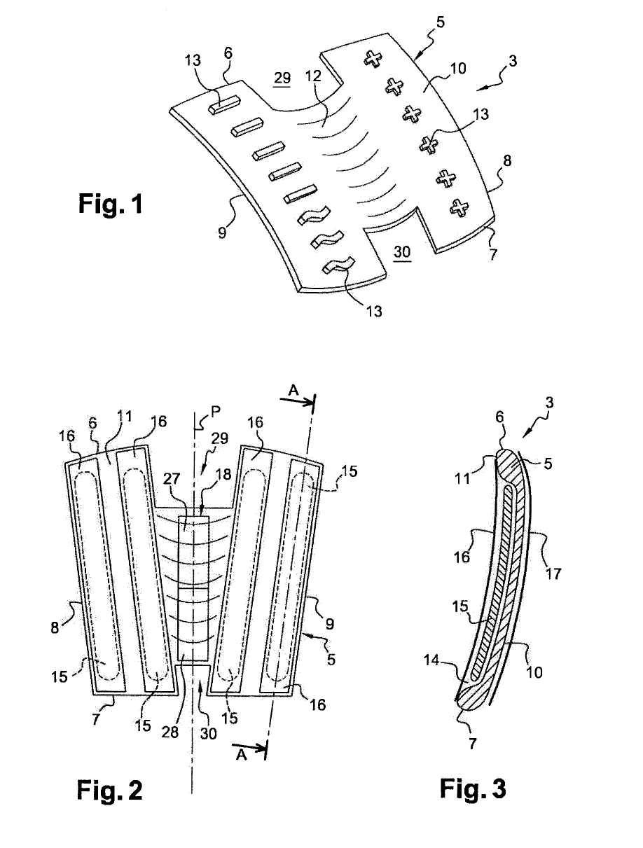

FIG. 1 is a perspective view of a dorsal element belonging to a kit according to the invention and seen from its internal side;

FIG. 2 is a plane view of the dorsal element of FIG. 1, seen from its external side;

FIG. 3 is a cross-sectional view of the dorsal element according to line AA of FIG. 2;

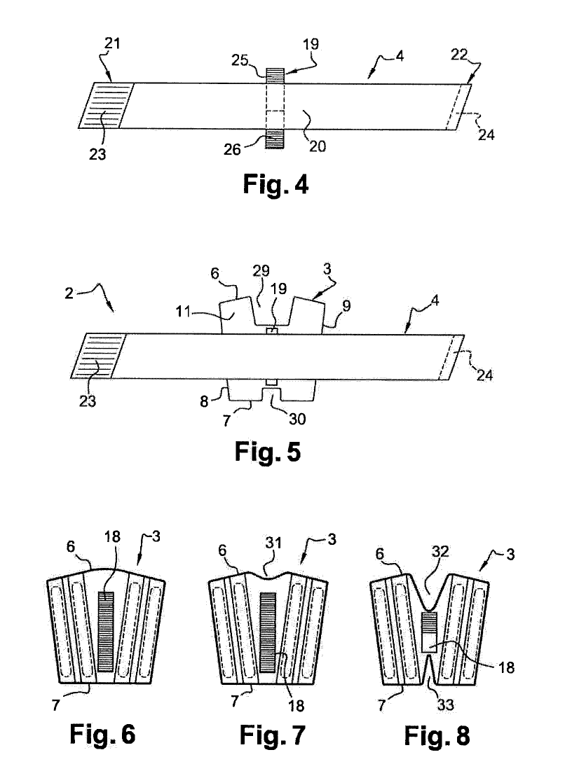

FIG. 4 is a plane view of a strip belonging to a kit according to the invention;

FIG. 5 is a plane view of a restraining belt formed by assembling the dorsal element of FIG. 1 and the strip of FIG. 4;

FIGS. 6 to 8 are plane views of alternatives of dorsal elements;

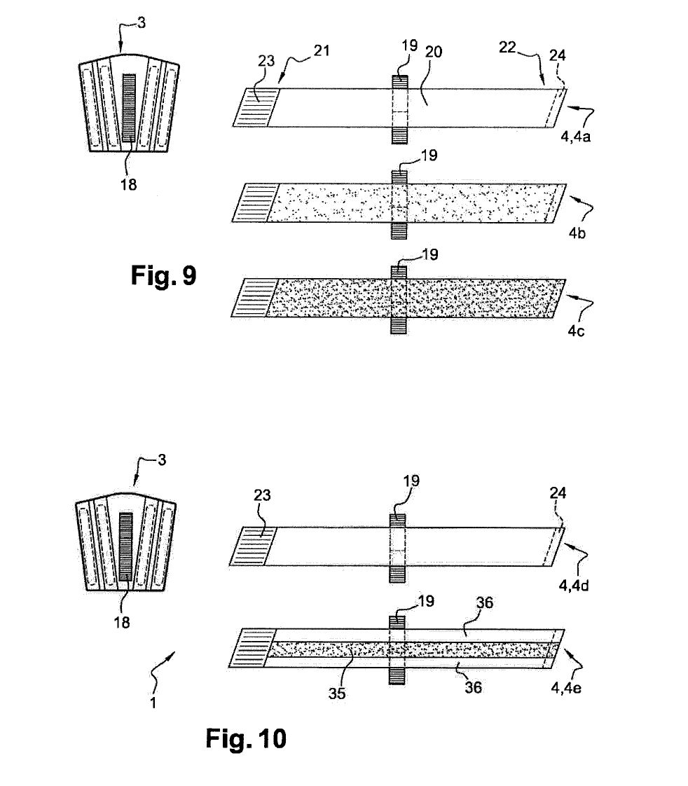

FIGS. 9 to 12 represent kits according to different embodiments of the invention.

The invention relates to a kit 1 for creating a lumbar support belt 2, the belt 2 being comprised of a dorsal element 3 intended to be placed against the lumbar region of an individual and a strip 4 able to be positioned around the lower portion of the trunk of the individual.

FIGS. 1 to 3 represent a dorsal element 3 according to an embodiment of the invention.

The dorsal element 3, which has a vertical plane of symmetry P as illustrated on FIG. 2, mainly comprises a body 5. The body 5 may be obtained by cutting then thermoforming a panel of material, for example a panel of foam such as polyester foam or polyamide foam. According to a possible embodiment, the body 5 is made of thermocompressible foam, that is to say that the geometry of the body 5 is not only obtained by deforming the panel which substantially maintains a constant thickness, but also by compressing certain areas, which leads to the formation of hollows and reliefs.

Advantageously, the body 5 is made from a breathable material—that is to say having sufficient permeability for water vapor—and/or is provided with orifices, such as to improve the breathability of the belt 2 and hence the comfort of the wearer.

The body 5 exhibits a suitable geometry and dimensions for the dorsal element 3 to press against the back of an individual by allowing a satisfactory comfort level.

Thus, the body 5 may exhibit in plane the general form of a rectangle, or preferably a trapezoid. It has, in use position, an upper edge 6 corresponding to the large base of the trapezoid, a lower edge 7 corresponding to the small base of the trapezoid, and two lateral edges 8, 9 slanted towards each other in the direction of the lower edge 7. By way of example, the height of the body 5 (between the upper edge 6 and the lower edge 7) may be in the range of 20 to 40 cm and the width (between the lateral edges 8, 9) in the range of 15 to 30 cm. The thickness of the body 5 is for example in the range of 3 to 8 mm.

Moreover, the body 5 has an internal side 10 turned towards the wearer, in use position, and an opposite external side 11.

The body 5 preferably has a double curvature to be better adapted to the anatomy of the wearer. Thus, it is curved from one lateral edge 8 to the other 9 as can be seen on FIG. 1, and from the lower edge 7 to the upper edge 6 as can be seen on FIG. 3, to follow the lordosis of the wearer.

Furthermore, it may be provided that the body 5 exhibits a groove 12 centered on plane P which, in use position, is substantially vertical and open inwards. The groove 12 is intended to be placed facing the vertebral column of the wearer, thus improving the wearing comfort. Another effect of the groove 12 is to ensure a good positioning of the dorsal element 3 on the wearer, but also to prevent its moving with respect to the wearer in a transversal direction.

The body 5 may also comprise on its internal side 10 reliefs 13 intended to limit the sliding of the dorsal element 3 with respect to the body of the wearer. Preferably, it is the sliding of the dorsal element 3 in the vertical direction that is sought to be avoided, this being very common when the wearer practices an activity or when he/she sits down. To this end, the reliefs 13 exhibit at least one portion oriented substantially transversally.

It may also be sought to prevent the sliding of the dorsal element 3 with respect to the body of the wearer in the transversal direction, in addition to the holding in this direction provided by the groove 12. To this end, the reliefs 13 exhibit at least one portion oriented substantially vertically.

As illustrated on FIG. 1, the reliefs 13 may take various shapes, and particularly the shape of horizontal lines, of “+” or wavy lines. The reliefs 13 are for example located along the two substantially vertical lines located on either side of the plane P.

The body 5 further exhibits cavities 14 opening onto its external side 11, which may be formed by localized deformation or compression of the material constituting the body 5. The cavities 14 are intended to receive a reinforcing member such as a stay 15. In the represented embodiment, the body 5 thus integrates four stays 15. A first set of stays 15, constituted of the two stays closest to the plane P, forms a V of low angle. Each of the stays 15 of a second set is parallel with one of the stays of the first set. In the embodiment represented, the stays 15 are substantially parallel with the lateral edges 8, 9 of the body 5. Preferably, the stays 15 are bent in order to match the shape of the lordosis of the wearer, as illustrated on FIG. 3.

The cavities 14 are blocked by a layer 16, for example comprising a piece of fabric, which is secured to the body 5 and thus forms with it a gusset wherein the stay 15 is maintained. This layer 16 may be secured to the body 5 by welding, sticking or sewing for example. The stays 15 are hence integrated to the dorsal element 3.

In the represented embodiment, each cavity 14 is blocked by a distinct layer 16, which is secured to the body 5 at the periphery of the cavity 14. It could be considered to provide a single layer 16 blocking the set of cavities 14 and defining the individual gussets for the stays 15.

The body 5 may also comprise one or several cavities, for example to receive a cushion serving to fill the lordosis. In this case, the cavity would open on the inner side of the body 5.

Furthermore, the dorsal element 3 preferably comprises a woven or unwoven material covering or surrounding the body 5. This material may be different on the internal side 10 and on the external side 11 as the needs differ. In fact, from the internal side, the material 17 substantially exhibits hygiene and comfort functions. From the external side, the function of the material—which may or may not be constituted of the layer 16—may be to allow the easy assembling of the dorsal element 3 with the strip 4. For example, it may thus consist in an astrakhan mesh or any other material allowing the assembling on Velcro® type hooks.

The dorsal element 3 is provided with assembly means 18 able to removably cooperate with complementary assembly means 19 formed on the strip 4 for creating the belt 2, wherein the strip 4 is assembled at the external side of the dorsal element 3 and tightly holds this dorsal element 3 against the body of the wearer.

As aforementioned, the assembling may be achieved by a Velcro® type system, the dorsal element 3 preferably bearing the loop type portion and the belt 4 the hook type portion, the opposite being nevertheless possible. The set of the external side 11 of the body 5 may be covered by a loop or velvet type material. Alternatively, as illustrated on FIG. 2, the assembly means 18 may be formed by a localized piece of velvet, astrakhan or equivalent, which is preferably located vertically along plane P.

According to another possible embodiment, the removable assembly means 18, 19 between the strip 4 and the dorsal element 3 may comprise a loop formed on the dorsal element 3 and wherein the strip 4 may be engaged.

The strip 4 represented on FIG. 4 is an elastic textile strip distinct from the dorsal element 3.

It comprises a central area 20 provided with assembly means 19 on the dorsal element 3. The latter are here exhibited in the form of a piece of material with hooks, turned inwards, able to cooperate with the assembly means 18 provided on the external side of the dorsal element 3. In the embodiment represented on FIG. 4, the assembly means 19 extend beyond the strip 4 on either side, in the vertical direction.

The strip 4 further comprises first and second extreme portions 21, 22 provided with complementary fastening means 23, 24 which cooperate to close the belt 2 when the first and second extreme portions 21, 22 of the strip 4 are placed on top of each other facing the abdominal region of the wearer.

The strip 4 may be of extremely simplified creation. In fact, the manufacturing to be carried out on the strip 4 may be limited to the placing of the fastening means 23, 24 and the assembly means 19.

The strip 4 has a certain number of structural characteristics, particularly in terms of nervousness and geometry. By geometry is meant the shape of the strip 4 and/or its dimensions.

Advantageously, the removable assembly means 18, 19 are arranged to allow an adjusting in height of the position of the strip 4 with respect to the dorsal element 3. This is particularly possible with a Velcro® system as described beforehand, the strip 4 being assembled on the dorsal element 3 at the desired height.

Furthermore, it may be provided that the removable assembly means 18, 19 comprise visual references able to indicate at least a relative positioning in height of the strip 4 with respect to the dorsal element 3. Thus, for example, the assembly means 18 of the strip 4 may comprise areas 25, 26 of different colors corresponding to the colors of areas 27, 28 provided on the assembly means 19 of the dorsal element 3. These visual references allow achieving the suitable positioning. It may consist of a single positioning, or the appropriate positioning from among several possible positionings, according to the needs or morphology of the wearer for example.

In the embodiment represented on FIGS. 1 to 4, the dorsal element 3 exhibits an upper notch 29 and a lower notch 30 which are substantially rectangular and centered on the plane P. The notches 29, 30 allow to facilitate a “folding” of the dorsal element 3 so that it is wound around the wearer to better match the shape of his/her lumbar region. Moreover, the notches 29, 30 provide visual centering points with respect to the vertebral column of the wearer.

Other geometries of the dorsal element 3 are nevertheless to be considered, and particularly:
the dorsal element 3 of FIG. 6 comprises a substantially rectilinear lower edge 7 and an upper convex edge 6, the dorsal element 3 being exempt of notches;
the dorsal element 3 of FIG. 7 comprises a substantially rectilinear lower edge 7 and an upper convex edge 6 provided with a central notch 31;
the dorsal element 3 of FIG. 8 exhibits an upper notch 32 and a lower notch 33 which are substantially triangular and centered on plane P.

Generally, it is possible to provide a dorsal element 3 which comprises a notch of suitable shape on its upper edge 6 and/or on its lower edge 7.

The kit 1 comprises a set of N strips 4 distinct from the dorsal element 3, with N≧2, each of the N strips having a certain number of structural characteristics. According to the invention, at least one structural characteristic is different from one strip to another within the set of N strips. As a result, the kit 1 allows to create a lumbar support belt 2 comprising said dorsal element 3 and a single strip 4 chosen from the set of N strips according to said desired structural characteristic.

A first embodiment of a kit 1 is illustrated on FIG. 9.

The structural characteristic differing from one strip to another within the set of N strips is the average nervousness of the strip 4. The strips (here, three) have more or less important average nervousness for providing a more or less important restraint. The N strips of the set exhibit for example the same nervousness at any point.

This kit allows creating a belt 2 which changes according to the recovery of the wearer, who will gradually be able to switch to lesser restraining degrees.

By way of example, it may be provided in the kit 1 a first strip 4a having a nervousness in the range of 350 to 400 cN/cm, a second strip 4b having a nervousness in the range of 450 to 500 cN/cm, and a third strip 4c having a nervousness in the range of 550 to 600 cN/cm.

A second embodiment of a kit 1 is illustrated on FIG. 10.

The structural characteristic differing from one strip to another within the set of N strips is the variation of nervousness in the strip 4.

For example, the kit 1 may comprise a first strip 4d exhibiting at any point the same nervousness and a second strip 4e having a central longitudinal area 35 exhibiting a first nervousness, allowing for suitable support for the lumbar region, and two extreme longitudinal areas 36 arranged on either side of the central area 35 and exhibiting a second nervousness lower than the first nervousness of the central area 35, to improve the wearing comfort at the ribs and the hips.

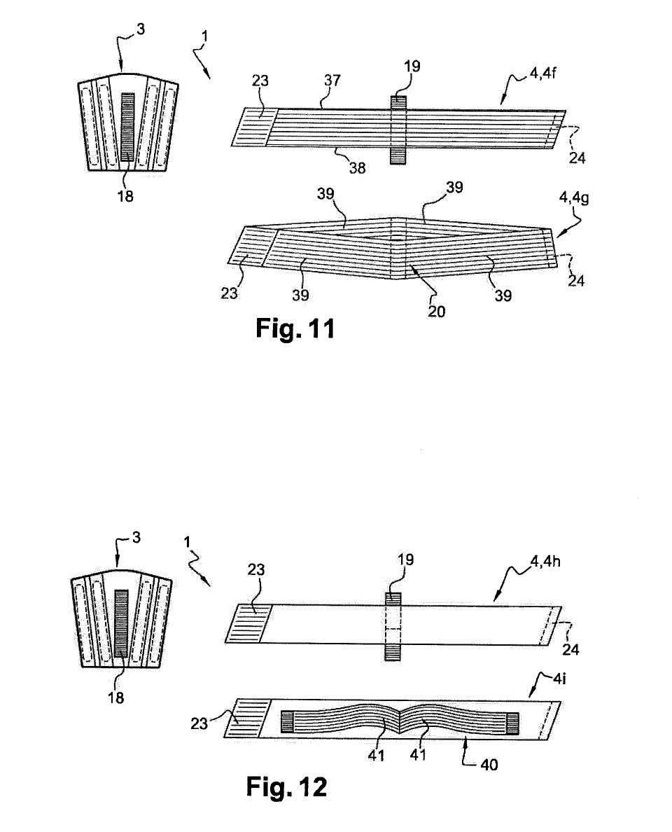

A third embodiment of a kit 1 is illustrated on FIG. 11.

The structural characteristic differing from one strip to another within the set of N strips is the geometry of the strip 4.

In the example represented, the kit 1 comprises two strips having different shapes. It may also be considered a kit 1 comprising strips 4 of identical shapes (for example substantially rectangular) but of different dimensions (for example more or less wide strips).

The kit 1 comprises here a first strip 4f having an upper edge 37 and a lower edge 38 substantially rectilinear and parallel. A second strip 4g comprises a widened central area 20. In order to simplify the manufacturing, the widened central area 20 may be obtained by superposing rectilinear portions 39 assembled in a diamond shape, for example rather than by cutting and binding of a single piece of textile.

The kit 1 thus allows to better adapt the belt 2 to the morphology of the wearer and to his/her activity, while providing as much comfort as possible. Thus, the wearer may prefer to wear a belt 2 with the first strip 4f when he/she is sitting, for example while driving, as a widened belt may bother him/her in the sitting position. On the other hand, when he/she is practicing a more sustained activity, it is advised to use a belt with a widened area in order to provide better restraint. Hence, this kit 1 allows creating a low cost multipurpose belt 1.

A fourth embodiment of a kit 1 is illustrated on FIG. 12.

The different structural characteristic from one strip to another within the set of N strips is the presence of an additional tightening device.

For example, the kit 1 comprises a first simple strip 4h, and a second strip 4i having an additional tightening device 40 in the form of two lateral straps 41. Thus, the strip 4i may not be overtightened and, when it is desired, it is possible to temporarily increase the tightening intensity, usually when the wearer is going to make an important effort, carry a heavy load, etc., by pulling the two lateral straps 41 and by fastening them to the strip 4i or one on top of the other by suitable fastening means. After, it is possible to return to the initial tightening, of lesser intensity, by detaching the additional tightening device.

It goes without saying that the invention is not limited to the aforementioned embodiments by way of examples but that it includes all the technical equivalents and alternatives of the means described as well as their combinations.

Thus, in the examples of aforementioned kits, the number of strips could be different from that represented and described. Furthermore, a kit could comprise strips having several structural characteristics that are different from the structural characteristics of the other strips of the kit. For example, it may be provided a kit comprising a first strip of constant nervousness, a second strip of higher nervousness and having in addition a widened central area, and a third strip having an additional tightening device and a variable nervousness.

Furthermore, if the kit 1 advantageously has a single dorsal element 3, the latter may be different from that represented on FIGS. 9 to 12. It may particularly be similar to one of those illustrated on FIG. 1, 7 or 8.

1-12. (canceled)

13. A kit for creating a lumbar support belt, comprising:
a dorsal element intended to be placed against the lumbar region of an individual, said dorsal element comprising at least two reinforcing members;
a set of N strips of elastic textile distinct from the dorsal element, where N≧2, each of the strips being able to be positioned around the lower portion of the trunk of the individual and comprising:
a central area provided with assembly means able to removably cooperate with complementary assembly means formed on the dorsal element;
first and second extreme portions provided with complementary fastening means.
each of the N strips having a certain number of structural characteristics, at least one structural characteristic differing from one strip to another within the set of N strips, the kit making it possible to create a lumbar support belt comprising said dorsal element and a single strip chosen from the set of N strips according to said structural characteristic desired.

14. The kit according to claim 13, wherein a structural characteristic differing from one strip to another within the set of N strips comprises the average nervousness of the strip.

15. The kit according to claim 13, wherein a structural characteristic differing from one strip to another within the set of N strips comprises the variation of nervousness in the strip.

16. The kit according to claim 13, wherein a structural characteristic differing from one strip to another within the set of N strips comprises the geometry of the strip.

17. The kit according to claim 13, wherein at least one of the strips within the set of N strips comprises an additional tightening device.

18. The kit according to claim 13, wherein the dorsal element comprises a body obtained by cutting then thermoforming a panel of material, for example a panel of foam.

19. The kit according to claim 13, wherein the dorsal element comprises a body made from a breathable material and/or provided with orifices.

20. The kit according to claim 13, wherein the dorsal element comprises at least one cavity for receiving a reinforcing member, said cavity opening onto the external side of the dorsal element and being blocked by a layer secured to the dorsal element.

21. The kit according to claim 13, wherein the dorsal element comprises on its internal side at least one relief intended to limit the sliding of said dorsal element with respect to the body of the wearer in at least one direction.

22. The kit according to claim 13, wherein the dorsal element exhibits a substantially vertical groove, in use position, intended to be placed facing the vertebral column of the wearer.

23. The kit according to claim 13, wherein the removable assembly means are arranged to allow adjusting the position of the strip in height with respect to the dorsal element.

24. The kit according to claim 13, wherein the removable assembly means comprise visual references able to indicate at least a relative positioning in height of the strip with respect to the dorsal element.
